(19) United States
(12) Patent Application Publication
(10) Pub. No.: US 2003/0215394 A1
(43) Pub. Date: Nov. 20, 2003

(54) Microparticles having a matrix interior useful for ultrasound triggered delivery of drugs into the bloodstream

(76) Inventors: Robert E. Short, Los Gatos, CA (US); Thomas B. Ottoboni, Belmont, CA (US); Matthew B. Kerby, Belmont, CA (US)

Correspondence Address: FISH & RICHARDSON P.C., 500 ARGUELLO STREET, SUITE 500, REDWOOD CITY, CA, 94063, US

(21) Appl. No.: 10/150,450

(22) Filed: May 17, 2002

(51) Int. Cl. A61K 9/14

(52) U.S. Cl. 424/9.52; 424/489

(57) ABSTRACT

A microparticle composition is provided for delivery of a pharmaceutical agent by ultrasound triggering. The microparticles have a porous, gas-containing interior polymer matrix and a plurality of cavities in the matrix which contain a gas and the agent. Methods for forming the microparticles and their use in ultrasonic diagnostic imaging and drug delivery are also provided.

### BACKGROUND

A variety of processes for the encapsulation of bioactive materials into microparticles have been developed over the years. The techniques have been optimized for many purposes including sustained release of drug over time, reduction in systemic drug toxicity, improved drug stability and site-specific drug delivery. The modalities have generally depended upon either the diffusion of the drug through the microparticle walls or the erosion of the encapsulating material. Methodologies developed for these functions are not applicable for indications where a mediated release of the entire payload at a predetermined site of delivery is required.

Diagnostic ultrasound provides a noninvasive means for imaging the internal structures of the human body. Early in its development there was the recognition that gas acts as a virtual mirror to ultrasound. This spurred the development of injectable gas-filled microparticles which could be used to enhance imaging of the cardiovascular system. Such microbubbles are sensitive to the insonate beam and could be ruptured and lose the gaseous core.

Accordingly, a gas-filled microparticle that is rupturable when exposed to ultrasound also has potential in applications where site-specific delivery of a drug is desired. Microparticles can be fabricated to encapsulate a drug as well as a gas. These microparticles can then be dispersed within the bloodstream and insonated with ultrasound at intensity sufficient to cause the microparticles to rupture thereby releasing the drug into the surrounding medium. Thus, the circulating microparticles do not release their drug payload until they are triggered to do so using ultrasound. For example, a drug may be selectively released in the heart by injecting a suspension of drug-containing gas-filled microparticles, allowing them to systematically circulate. Then an ultrasound beam can be focused at the heart to rupture the microparticles entering the heart. This type of drug delivery system is particularly advantageous when toxicity issues arise from systemic delivery of a drug. By limiting release of a pharmaceutical agent to a specific targeted site, toxic side effects can be minimized. In addition, total required dosage will typically be lower and result in a decrease in costs for the patient.

The use of gas-filled ultrasound contrast agents serving also as drug carriers has been described for gas-filled liposomes in U.S. Pat. No. 5,580,575. A quantity of liposomes containing drug is administered into the circulatory system of a patient and monitored using ultrasonic energy at diagnostic levels until the presence of the liposomes are detected in the region of interest. Ultrasonic energy is then applied to the region at a power level that is sufficient to rupture the liposomes thus releasing the drug. The ultrasonic energy is described in U.S. Pat. No. 5,558,082 to be applied by a transducer that simultaneously applies diagnostic and therapeutic ultrasonic waves from transducer elements located centrally to the diagnostic transducer elements.

The use of gas-filled microcapsules to control the delivery of drugs to a region of the body has also been described in U.S. Pat. No. 5,190,766 in which the acoustic resonance frequency of the drug carrier is measured in the region in which the drug is to be released and then the region is irradiated with the appropriate sound wave to control the release of drug. Separate ultrasound transducers are described for the imaging and triggering of drug release in the target region.

### SUMMARY

The invention provides a microparticle composition for delivery of a pharmaceutical agent by ultrasound triggering comprising microparticles having a porous gas-filled polymer matrix interior and a plurality of hollow cavities dispersed within the matrix containing a gas and the pharmaceutical agent. The microparticles may optionally have outer shells of a polymer that is distinct from the polymer matrix interior. The gas may be air, nitrogen, oxygen, argon, helium, carbon dioxide, xenon, a sulphur halide, a halogenated hydrocarbon or combinations thereof.

A method is also provided of forming a microparticle composition suitable for delivering a pharmaceutically active agent by ultrasonic triggering comprising the steps of:

a. forming a first emulsion from a first aqueous phase containing a pharmaceutically active agent and an organic solvent phase containing a polymer immiscible or largely immiscible with the aqueous phase;

b. forming a second emulsion from the first emulsion and a second aqueous phase to form droplets containing the organic solvent phase and a plurality of droplets of the first aqueous phase;

c. removing the solvent from the organic solvent phase and the water from the first aqueous phase to form microparticles having a porous gas-filled polymer matrix interior and a plurality of hollow cavities dispersed within the matrix.

An outer shell around the microparticles can be formed by using an organic solvent phase containing a second solvent and a second polymer soluble in the solvent mixture and insoluble in the first solvent. Upon removal of the second solvent after step b) an outer shell is formed from the second polymer.

An outer layer of a biologically compatible amphiphilic material may be formed by using a second aqueous phase containing the biologically compatible amphiphilic material. Upon diluting the second emulsion with an aqueous bath containing a chemical cross-linking agent after step b) an outer layer is formed on the microparticles.

Also provided is a method for delivery of a pharmaceutical agent to a region of interest within a fluid filled cavity, vessel, or fluid perfused tissue by ultrasound triggering comprising the steps of:

a. introducing the microparticle composition into the region of interest;

b. applying an ultrasound signal to the region of interest at a power intensity sufficient to induce rupture of the microparticles;

c. maintaining the power intensity until at least a substantial number of the microparticles are ruptured.

### DESCRIPTION OF DRAWINGS

FIG. 1 is an illustration of a cross-sectional view of a microparticle according to the present invention.

FIG. 2 is a graph of the wavelength vs. absorbance showing increased dye release from microparticles due to insonation according to the procedure described in Example 5.

### DETAILED DESCRIPTION

This invention pertains to novel microparticle compositions which are suitable as intravenous drug carriers that are triggered to readily release the drug upon insonation at ultrasonic frequencies and power commonly employed by diagnostic imaging devices. Such compositions are useful in applications requiring a noninvasive means of delivering drug to a local site while limiting systemic exposure.

The present invention provides compositions of microparticles for delivery of pharmaceutical agents wherein each particle comprises a gas-filled polymeric matrix interior and a plurality of cavities dispersed within the matrix. The cavities contain a gas and a pharmaceutical agent. The microparticles may optionally have an outer shell composed of a polymeric material distinct from the polymeric matrix interior. The microparticles may also optionally have an outer layer chosen separately on the basis of biocompatibility with the bloodstream and tissues. The materials and amounts thereof comprising the matrix interior and the optional outer shell may be selected to predetermine the strength of the microparticle. For example, strength may be predetermined to provide a desired threshold of ultrasound power at which the microparticle ruptures to release its contents. Alternatively, the material for the optional outer layer may be selected as to provide versatility in modifying charge or chemistry of the microparticle surface without affecting the acoustic properties of the microparticle. Methods for forming the microparticles and their use in ultrasonic diagnostic imaging and drug delivery are also provided.

As used herein, the term microparticle refers to a particle of approximately spherical shape. The microparticles are typically within the size range of 1 and 1000 microns. It is not necessary for the microparticles to be precisely spherical although they generally will be spherical and described as having average diameters. If the microparticles are not spherical, then their diameters are linked to the diameter of a corresponding spherical microparticle enclosing approximately the same volume of the interior space as the non-spherical microparticle.

The microparticles according to the present invention are composed of a polymer matrix interior containing a plurality of cavities. Contained within the cavities is a pharmaceutical agent. The interior matrix of the microparticle comprises a biodegradable polymer which may be tailored to provide the desired drug-accommodating and acoustic properties. The biodegradable polymer may be a naturally occurring biopolymer or a synthetic polymer.

The polymer matrix interior also contains a gas within the void spaces of the porous structure. It is this gas that renders the microparticles rupturable by ultrasound energy. When the microparticles are suspended in an aqueous medium, the gas will be retained in the interior due to the hydrophobicity of the microparticle surface. When exposed to an insonate beam at frequencies and power levels typical of diagnostic ultrasound equipment, the microparticles will flood. While not intending to be bound by any particular theory, it is believed that the ultrasonic wave forces an oscillation of the gas-filled microparticles. As the microparticles oscillate, a pressure differential is created which overcomes the hydrophobic tensile forces allowing the surrounding aqueous medium to wick into the microparticle.

The degree of porosity of the interior matrix will depend upon the application and will typically have a void-to-polymer ratio within the range of 30-95%. By varying the void volume the acoustic properties of the microparticles may be tailored. Relatively less void volume renders microparticles more resistant to rupture by ultrasound energy while microparticles having a relatively greater void volume are more fragile and thus less resistant to being ruptured.

Selection of polymer comprising the matrix interior will affect the mechanical and acoustic properties of the microparticles. For example, those materials having a higher yield stress property provide a less fragile microparticle. Such a population of microparticles would thus require a relatively higher ultrasound power level to release the drug contents. Average molecular weight of the material may also be manipulated to modify the properties of the microparticles. A lower molecular weight polymer generally produces a more easily rupturable microparticle. Use of additives such as plasticizers may also typically affect the mechanical properties of the material including its yield strength.

The hollow cavities or vesicles contained within the matrix interior of the microparticle, while also containing mostly gas, are structural entities which are distinct from the void spaces of the matrix. While not intending to limit the location of the drug to one particular location within the microparticle, these vesicles are the primary receptacle for the pharmaceutical agent. When the microparticle is ruptured or otherwise made to flood, the surrounding aqueous medium wicking into the interior will also flood the drug containing vesicles. The payload within then dissolves and the solution will freely diffuse into the surrounding medium.

The drugs typically applicable for ultrasound triggered delivery are, for example, cardiovascular drugs (endocardium agents) with short circulatory half-lives that affect the cardiac tissues, vasculature and endothelium to protect and treat the heart from ischemic or reperfusion injury or coronary artery from restenosis (anti-restenosis agent). Drugs which target platelets (anti-platelet agent) and white cells (anti-white cell agent) which may plug the microvasculature of the heart after a heart attack are also useful for local cardiac delivery. Another type of drug is one for which a local effect is required but where systemic effects of the drug would be detrimental. These are typically drugs with high toxicity, for example, locally administered potent vasodilators which increase blood flow to hypoxic tissue. If delivered systemically these would cause a dangerous drop in blood pressure. Suitable drugs include fibronolytic agents such as tissue plasminogen activator, streptokinase, urokinase, and their derivatives, vasodilators such as verapamil, multifunctional agents such as adenosine, adenosine agonists, adenosine monophosphate, adenosine diphosphate, adenosine triphosphate, and their derivatives, white cell or platelet acting agents such as GPllb/llla antagonists, energy conserving agents such as calcium channel blockers, magnesium and beta blockers, endothelium acting agents such as nitric oxide, nitric oxide donors, nitrates, and their derivatives, free-radical scavenging agents, agents which affect ventricular remodeling such as ACE inhibitors and angiogenic agents, and agents that limit restenosis of coronary arteries after balloon angioplasty or stenting.

In addition to therapeutic agents delivered locally to the heart, the use of vasodilators in the microparticles have diagnostic application. Vasodilators are used in cardiology to assess the coronary blood flow reserve by comparing blood flow in the heart with and without the maximal vasodilation by the pharmaceutical agent. Coronary blood flow reserve correlates well with patient prognosis since the reserve capacity enables the myocardium to remain viable during a heart attack. Adenosine and other vasodilators are used during interventional cardiology and nuclear imaging to determine coronary reserve. A microparticle agent which contains a vasodilator is useful in echocardiography to examine the myocardium under normal conditions, and then, upon release of the vasodilator by the ultrasound beam, under conditions to stimulate local vasodilation. The coronary blood flow reserve may be estimated non-invasively using ultrasound imaging by the extent of hyperemia of the myocardium, Doppler regional flow, or by other well known methods of characterizing the ultrasound imaging data.

Another class of therapeutic moieties deliverable by microparticles triggered by ultrasound is chemotherapeutic agents used for the treatment of various cancers. Most of these agents are delivered by intravenous administration and can produce significant systemic side effects and toxicities that limit their dose and overall use in the treatment of cancer. For example, doxorubicin is a chemotherapy drug indicated for the treatment of breast carcinoma, ovarian carcinoma, thyroid carcinoma, etc. The use of doxorubicin is limited by its irreversible cardiotoxicity, which may be manifested either during, or months to years after termination of therapy. Other side effects commonly associated with chemotherapeutic agents include hematologic toxicity and gastrointestinal toxicity. For example, carmustine is associated with pulmonary, hematologic, gastrointestinal, hepatic, and renal toxicities. The utility of doxorubicin, carmustine, and other chemotherapy agents with a narrow therapeutic index may be improved by delivering the drug at the tumor site in high concentrations using ultrasound-triggered microparticles while reducing the systemic exposure to the drug.

The gas contained within the microparticle may be any non-toxic gas and may be selected on the basis of the acoustic and drug-dispensing properties required of the microparticle for the application. The gases are typically air, nitrogen, oxygen, argon, helium, carbon dioxide, xenon, a sulfur halide, a halogenated hydrocarbon and combinations of these. It is known that different gases have different solubilities in the blood. Carbon dioxide, for example, has a high solubility. Thus, a microparticle containing carbon dioxide will lose its gas rapidly and therefore will have a corresponding payload release rate. Alternatively, perfluorocarbon gases, such as sulfur hexafluoride or perfluorobutane, slowly dissolve. Microparticles containing such a gas will release the payload at a relatively slower rate. Nitrogen and oxygen have intermediate solubilities and therefore the release rates would be correspondingly intermediate.

In another embodiment of the invention, the microparticles may also comprise an outer polymer shell comprising a material that is distinct from the inner polymer matrix. Since the shell is formed from a different material, the structure may be tailored separately to modify the microparticle acoustic or drug dispensing properties. For example, a thicker, less porous wall will act to increase the microparticle acoustic strength and retard drug release. This outer shell material may be selected from the same polymers suitable for use in the inner polymer matrix.

The microparticles may optionally comprise an outer layer made of a biocompatible material. The outer layer material will typically be amphiphilic, that is, have both hydrophobic and hydrophilic characteristics. Such materials have surfactant properties and thus tend to be deposited and adhere to interfaces, such as the outer surface of the microparticle or the microparticle precursor. Preferred materials are biological materials including proteins such as collagen, casein, gelatin, serum albumin, or globulins. Human serum albumin is particularly preferred for its blood compatibility. Synthetic polymers such as polyvinyl alcohol may also be used.

Provision of a separate outer layer allows for charge and chemical modification of the surface of the microparticles, particularly if the material for the matrix interior or optional outer shell is not readily modifiable for such purpose. Surface charge can be selected, for example, by providing an outer layer of a type A gelatin having an isoelectric point above physiological pH or by using a type B gelatin having an isoelectric point below physiological pH. The outer surface may also be chemically modified to enhance biocompatibility, such as by pegylation, succinylation or amidation, as well as being chemically binding to the surface-targeting moiety for binding to selected tissues. The targeting moieties may be antibodies, cell receptors, lectins, selecting, integrins, or chemical structures or analogues of the receptor targets of such materials.

If the drug delivery application requires that the microparticles be introduced into the vascular system, then it is preferred that majority of those in the population will have diameters within the range of about 1 to 10 microns. This will insure that the microparticles are small enough to pass through the capillary system unimpeded.

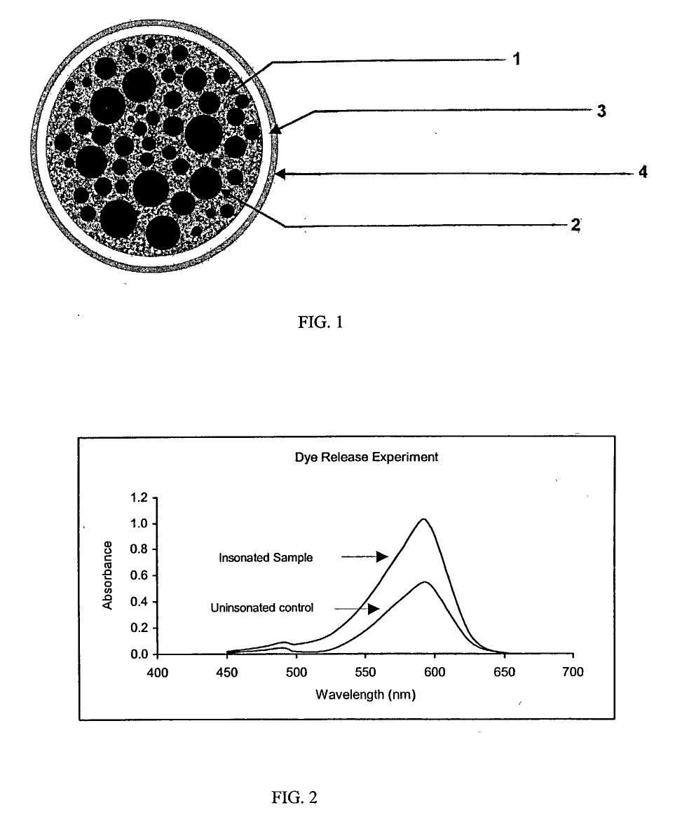

Referring to FIG. 1, there is shown a cross-sectional view of a microparticle representation according to the invention. The microparticle comprises a gas-filled polymer matrix (1), in which is dispersed drug-containing hollow vesicles (2), also containing a gas. Also depicted in the illustration are the optional outer polymer shell (3) and the optional biocompatible outer layer (4).

A method for the preparation of the matrix microparticles of the invention comprises a multiple phase emulsion technique with a variation from conventional procedure. Rather than evaporation, the polymer solvent is removed by lyophilization.

Typically, particle fabrication procedures using emulsion systems rely on evaporation of the polymer solvent to form the microparticle. Such a system does not normally result in a porous matrix structure. With evaporation, when the solvent undergoes phase change from liquid to vapor, the polymer molecules remain mobile within the liquid phase until the solvent is removed. Because of this mobility, surface tension forces draw the polymer molecules together to cohere and form an essentially void free solid mass.

By contrast, removal of the polymer solvent by lyophilization renders a polymer matrix construct which contains interstitial void spaces. Using lyophilization, or freeze-drying, the liquid is first frozen and then removed by sublimation in vacuo. When the solvent is frozen, the polymer molecules become fixed in place. Removal of the solvent by means of sublimation does not permit the polymer molecules to appreciably shift their relative position.

The first step in the fabrication process of the matrix microparticle is the preparation of the aqueous primary phase (W1). This involves the dissolution of the drug payload into an aqueous solution. Alternatively, with drugs having limited water solubility, solid drug particles may be dispersed within the primary aqueous phase as long as the particles are not readily soluble in the organic phase (solvent) and are of a size range consistent with the dimensions of the matrix microparticle construct. If the drug particles are appreciably solvent soluble, it is likely that the drug would partition into the organic phase during the emulsion process, incorporate into the polymer matrix, and thus restrict its dissolution into the surrounding medium. Preferably, the payload particulates will be small, i.e., less than one micron average diameter and well dispersed within the aqueous primary phase.

The primary aqueous phase may contain a surface active component to enhance microdroplet formation during the first emulsion process. Any number of hydrophilic surfactants would be suitable including the poloxamers, tweens, or the brijs. Also suitable are soluble proteins such as gelatin or albumin, or synthetic soluble polymers such as polyvinyl alcohol. Human serum albumin is particularly useful as the surface active component since it is additionally useful in reducing the deactivation of sensitive proteinaceous drugs which often occurs during encapsulation processes.

Addition of a viscosity enhancer to the primary aqueous phase may also be beneficial as an aid in stabilizing the emulsion. Such materials which may be useful in this regard include carboxymethyl cellulose, dextran, carboxymethyl dextran, hydroxyethyl cellulose, gum arabic, polyvinyl pyrrolidone, xanthan gum, hydroxyethyl starch, sodium alginate, and the like.

Optional components in the W1 phase include ingredients to balance osmolality with the outer aqueous phase and stabilizers to preserve drug efficacy during the lyophilization phase of the process and during storage.

The drug containing primary aqueous phase is then emulsified into a middle phase organic solvent-polymer solution (O) to make a W1-O emulsion. It is this middle phase which forms the matrix construct of the microparticle. The ratio of primary aqueous phase to oil phase should be less than about 1:2, with about 1:5 being preferred. Higher ratios may tend to become bicontinuous or may invert to an O-W emulsion.

A variety of devices can be used to produce the emulsion, e.g., colloid mills, rotor/stator homogenizers, high pressure homogenizers, and ultrasonic homogenizers. Sonication using an ultrasonic homogenizer is most preferred in producing the primary emulsion.

Preferably, the matrix forming polymer is biocompatible and, more preferably, bioabsorbable. Examples include polylactide, polyglycolide, polycaprolactone, polyhydroxybutyrate, polyhydroxyvalerate or copolymers of two or more of them, copolymers of lactides and lactones, polyalkylcyanoacrylates, polyamides, polydioxanones, poly-beta-aminoketones, polyanhydrides, poly (ortho) esters, and polyamino acids. A preferred polymer is polylactide.

The polymer solvent can be any solvent which is capable of dissolving the matrix forming material, is substantially immiscible with water, and is lyophilizable. By lyophilizable, it is meant that the solvent will freeze at a temperature well above the temperature of the lyophilizer condenser and that the frozen solvent will sublimate at a reasonable rate in vacuo. Suitable solvents include p-xylene, benzene, benzyl alcohol, hexanol, decane, undecane, tetradecane, cyclohexane, cyclooctane and the like.

The concentration of polymer dissolved in the solvent can vary from about 0.5% to 20% by weight or greater. A more fragile microparticle construct is achieved at lower concentrations while a more durable microparticle is provided for by using a higher concentration.

The use of a co-solvent with the above solvent is not precluded so long as it is also lyophilizable or is otherwise removed prior to the lyophilization process. The addition of a co-solvent may advantageously modify the characteristics of the primary emulsion. For example, the flocculation of the primary aqueous phase droplets can be reduced by the inclusion of a co-solvent. The co-solvent may be substantially miscible with water, such as dioxane, acetone, or tetrahydrofuran, or immiscible with water, such as isopropyl acetate, methylene chloride, or toluene.

Use of a second co-solvent is typical when the formation of the optional shell wall is desired. In this case, the second co-solvent is removed prior to lyophilization. With such a procedure, the wall forming polymer/co-solvent system is selected such that the wall forming polymer is soluble in the solvent mixture but is essentially insoluble in the first solvent. Thus, when the second solvent is removed, by evaporation, for example, then the wall forming polymer precipitates at the emulsion interface to form a polymer shell around the droplet.

Advantageous characteristics may be imparted to the microparticles by the addition of modifiers to the middle phase. For example, addition of a compatible plasticizer may reduce the elastic modulus of the polymer and thereby change the acoustic properties of the microparticle. Dissolution of a wax or a fatty acid in the middle phase may render the microparticle more hydrophobic and thus more resistant to flooding.

The primary W1-O emulsion is then added to a secondary aqueous phase (W2) and this mixture in turn is emulsified to form the secondary emulsion (W1-O-W2). This second emulsification step generates organic polymer droplets containing smaller droplets of the primary aqueous phase. The secondary aqueous phase preferably contains surface active components to enhance emulsification of the primary emulsion. Any number of hydrophilic surfactants are suitable including the poloxamers, tweens, or the brij's. Also suitable are soluble proteins such as gelatin, albumin, globulins, and casein and synthetic polymers such as polyvinyl alcohol.

Addition of a viscosity enhancer to the secondary aqueous phase may also be beneficial as an aid in stabilizing the emulsion. Such materials which may be useful include carboxymethyl cellulose, dextran, carboxymethyl dextran, hydroxyethyl cellulose, gum arabic, polyvinyl pyrrolicine, xanthan gum, hydroxyethyl starch, sodium alginate, and the like.

The range of ratios of the primary emulsion, W1-O, to the secondary aqueous phase, W2, is between about 2:1 and 1:20. Preferred is a ratio of about 1:1. It is advantageous that the secondary aqueous phase be osmotically balanced with the primary aqueous phase. If the W2 phase is not isotonic to W1, then the imbalance can cause a net diffusion of water across the organic polymer middle phase which will affect the volume and contents of the W1 phase.

The size of the droplets formed from the secondary emulsion should be in a range that is consistent with the application. For example, if the microparticles are to be injected intravenously, then they should have diameters of less than 10 microns in order to pass through the capillary network unencumbered. The types of equipment that may be used to produce the secondary emulsion are the same as those used to form the primary emulsion although the shearing forces will be much reduced. High or prolonged shearing of the mixture increases the likelihood of losing the primary aqueous phase from the interior of the middle phase droplets. The secondary emulsion may be satisfactorily formed using a rotor/stator homogenizer, but microporous membrane homogenization techniques are preferred since there is more uniform shearing to produce a more monodisperse population of droplets. Membrane homogenization involves pumping a pre-emulsion through a porous material, such as a sintered glass or metal element to more finely divide the discontinuous phase droplets.

To provide the optional outer polymer shell, a second wall-forming polymer is additionally dissolved in the organic middle phase. The wall-forming polymer may be selected from the same polymers suitable for use as the polymer matrix interior, provided it is not identical to it in a given population of microparticles. Then the second polymer is made to precipitate at the outer surface of the organic droplet (O). This may be achieved by several means. One method is to select the wall-forming polymer and the solvent such that the polymer remains in solution as long as the system is maintained within a specified temperature range but is then precipitated when the solution is brought to a temperature outside the specified range. A second method is to utilize a co-solvent system as described earlier such that the wall-forming polymer remains soluble until one of the solvents of the co-solvent system is substantially removed by evaporation or other means. In either case, the first polymer which forms the inner polymer matrix remains soluble during the formation of the shell. A preferred wall-forming polymer is polylactide-co-glycolide.

Optionally, the W1-O-W2 emulsion can be diluted into a larger aqueous bath. This optional step is useful when, for example, a stabilized protein outer layer to the middle phase droplet is to be provided. Because many proteins are amphiphilic and thus surface active, a portion will adsorb to the surface of the droplet, forming an outer protein layer around it. A chemical crosslinker such as an aldehyde or a carbodiimide added to the larger aqueous bath will stabilize the protein on the surface. This outer protein coat is desirable, for example, if an application requires a tailored bioreactive microparticle surface.

It may be desirable to further modify the surface of the microparticle, for example in order to passivate the surface against macrophages or the reticuloendothelial system (RES) in the liver. This may be accomplished by chemically modifying the surface of the microparticle to be negatively charged since negatively charged particles appear to better evade recognition by macrophages and the RES than positively charged particles. Also, the hydrophilicity of the surface may be changed by attaching hydrophilic conjugates, such as polyethylene glycol (pegylation) or succinic acid (succinylation) to the surface, either alone or in conjunction with the charge modification. The protein surface may also be modified to provide targeting characteristics for the microparticle. The surface may be tagged by known methods with antibodies or biological receptors.

Optionally, the organic-polymer droplets are rinsed and concentrated. This can be achieved by centrifugation or by diafiltration. The rinsing solution should be osmotically balanced with the primary aqueous phase contained within the droplet to eliminate any concentration gradient that would drive the diffusion of water across the organic-polymer boundary.

The polymer-organic droplets may then be formulated with excipients and then lyophilized. The suspending medium preferably contains ingredients to inhibit droplet aggregation, such as surfactants. Bulking agents and cryoprotectants are also preferably included in the suspending medium. Typical bulking agents are sugars such as mannitol, sucrose, trehalose, lactose, and sorbitol and water soluble polymers such as polyethylene glycol, polyvinyl pyrrolidone, and dextran.

It is desirable that the osmolality of the reconstituted lyophilized microparticle suspension be physiologically isotonic. The bulking agents utililized during the lyophilization of the microparticles may be used to control the osmolality of the final formulation for injection. Alternatively, additional ingredients may be added to the excipient formulation such as buffering salts or amino acids to balance the osmolality.

During the lyophilization process the polymer solvent and both the water of the W1 and the water of the excipient suspending medium are removed at reduced pressure by sublimation to form a population of substantially solvent free microparticles having a polymer matrix interior. Within the matrix are the hollow cavities formed by the sublimation of the frozen primary aqueous phase. The drug payload will remain in the hollow cavities until the microparticle is made to rupture in the bloodstream using ultrasound.

In clinical use, the dry lyophilized product may be reconstituted by addition of an aqueous solution and the resulting microparticle suspension intravenously injected. As the microparticles circulate systemically, their presence at the site of delivery can be monitored using an ultrasound device operating at power levels below that which is required to rupture the microparticles. Then at the appropriate time, when a required concentration of microparticles is present at the site, the power level can be increased to a level sufficient to rupture the microparticles, thus triggering the release of the drug payload.

Preferably, the rupture of the drug-carrying microparticles is achieved using ultrasound scanning devices and employing transducers commonly utilized in diagnostic contrast imaging. In such instances a single ultrasound transducer may be employed for both imaging and triggering of the microbubbles by focusing the beam upon the target site and alternately operating at low and high power levels as required by the application.

Alternatively, a plurality of transducers focused at the region may be used so that the additive wave superposition at the point of convergence creates a local intensity sufficient to rupture the microparticles. A separate imaging transducer may be used to image the region for treatment.

While not required, it is preferred that the microparticles be rupturable for drug release at power levels below the clinically accepted levels for diagnostic imaging. Specific matching of ultrasound conditions and microparticle response to such conditions achieve controlled release conditions. Preferred acoustic conditions for rupture are those at a power, frequency, and waveform sufficient to provide a mechanical index from about 0.1 to about 1.9.

The following examples are provided by way of illustration and are not intended to limit the invention in any way.

### EXAMPLE 1

### Encapsulation of HSA in a Polymer Matrix Microparticle

A solution of 5% human serum albumin (HSA) was prepared by dilution from a 25% HSA solution. A polymer solution of 5% wt/vol. was prepared using poly(DL-lactide) and p-xylene. One part 5% HSA solution was slowly added to 4 parts polymer solution while the mixture was continuously homogenized using a Virtis Virsonic ultrasonic homogenizer at a setting of 5. After all of the HSA solution was incorporated, the emulsion was further homogenized at power level 9 for 30 seconds. Microscopic examination of this primary emulsion revealed sub-micron size aqueous droplets that were well dispersed throughout the emulsion.

The primary emulsion was slowly added to an equal volume of 5% HSA solution at pH 7 with mixing using a 10 mm rotor-stator homogenizer. After all of the primary emulsion was added, the homogenizer was run at full power for 30 seconds. Examination of the secondary emulsion under a microscope showed discrete organic droplets containing microdroplets of the primary emulsion within.

The emulsion was diluted into an aqueous bath containing 0.25% glutaraldehyde at 40° C. After 5 minutes, poloxamer 188 surfactant was dissolved into the bath at a concentration of 0.25% to inhibit aggregation of the microparticles. A 50 ml sample of the bath was centrifuged at 2000 rpm for 10 minutes. The concentrated microdroplets were separated from the underlying liquid and then lyophilized in 10 ml vials containing an aqueous medium. When the drying cycle was completed, the lyophilization chamber was filled with nitrogen gas to a pressure of slightly less than atmospheric and the vials were then stoppered.

Microscopic examination of a reconstituted sample showed the microparticles were gas filled and roughly spherical. They were observed to readily float confirming that they were gas filled. The internal regions of the microparticles were not visible. The microparticles were observed to remain air filled 72 hours following reconstitution.

### EXAMPLE 2

### Encapsulation of Adenosine crystals in a Polymer Matrix Microparticle

A solution of 5% human serum albumin (HSA) was prepared by dilution from a 25% HSA solution. The 5% HSA solution (W1) was osmotically adjusted to 300 mOs/kg using dextrose. Separately, a polymer solution of 5% wt/vol. was prepared using poly(DL-Lactide) and an 85:15 mixture of p-xylene and isopropyl acetete as the solvent.

Separately, adenosine crystals were prepared by adding spray-dried adenosine powder into a 5% solution of poly(DL-Lactide) and isopropyl acetate. The spray-dried adenosine recrystalized into small particles averaging approximately 2 micron in size. The isopropyl acetate/polymer solution of the adenosine particle suspension was then exchanged with the 85:15 xylene:isopropyl acetate/polymer solution. The crystals were clearly observable in polarized light and well dispersed.

One part W1 solution was slowly added to 4 parts polymer solution containing the dispersion of adenosine crystals while ultrasonically agitating the organic solution at power level 5 using a Virtis Virsonic ultrasonic homogenizer. After all of the W1 solution was incorporated, the emulsion was further homogenized at power level 9 for 30 seconds. Microscopic examination of this primary emulsion revealed sub-micron sized water droplets that were well dispersed throughout the emulsion. The adenosine crystals remained well dispersed in the polymer solution.

The primary emulsion was slowly added to an equal volume of 5% HSA solution at pH 7 and a dextrose adjusted osmolality of 300 mOs/kg. A Pro Scientific 400 rotor-stator homogenizer with a 30 mm head, running at 2 k rpm was used during the addition to homogenize the sample. After all of the primary emulsion was added, the speed of the rotor-stator was increased to 6 k rpm for 45 seconds. Examination of the secondary emulsion under a microscope showed discrete organic drops containing smaller droplets of 5% HSA and adenosine crystals. Under polarized light conditions, the adenosine crystals were clearly visible as encapsulated within the organic-polymer droplets of the secondary emulsion. The organic-polymer droplets were retrieved, formulated in an osmotically balanced medium containing cryoprotectants and bulking agents, dispensed into 10 ml vials, and lyophilized. When the lyophilization cycle was complete, the lyophilization chamber was filled with nitrogen gas to a pressure of slightly less than atmospheric and the vials were then stoppered.

The lyophilized product was reconstituted with DI water. Microscopic examination of reconstituted microparticles clearly revealed that they were air filled. The microparticles were opaque and thus adenosine crystals were not visible. Dispersing the lyophilized product in oil caused some of the particles to flood. Using polarized light, the adenosine crystals could be clearly seen in many of the flooded microparticles.

### EXAMPLE 3

### Encapsulation of Bromophenol Blue Dye in a Polymer-Matrix Microparticle

A solution of 10% human serum albumin (HSA) was prepared by dilution from a 25% HSA solution. A 10% bromophenol blue dye solution was prepared and the pH was adjusted to 7 with sodium hydroxide. These two solutions were combined in equal parts to produce a 5% HSA and 5% bromophenol blue solution at pH 7. The solution osmolality was measured and adjusted to 300 mOs/kg using dextrose. This solution will be referred to as the W1 solution.

Separately, a 5% wt/vol. polymer solution was prepared with poly(DL-Lactide) using a mixture of p-xylene and isopropyl acetate in an 85:15 ratio. One part W1 solution was slowly added to 4 parts polymer solution while ultrasonically homogenizing the organic solution at power level 5 with a Virtis Virsonic ultrasonic homogenizer. After all of the W1 solution was added, the emulsion was further homogenized at power level 9 for 30 seconds. Microscopic examination of this primary emulsion revealed sub-micron size water droplets that were well dispersed throughout the emulsion.

An outer water phase (W2), consisting of a 5% HSA solution at pH 7, was prepared. The osmolality of this W2 solution was adjusted to 300 mOs/kg using dextrose to match the osmolality of the inner W1 phase. The W2 solution was placed in a 250 ml water-jacketed beaker maintained at 25° C. and agitated slowly with a stir bar. The primary water-in-oil emulsion was slowly added to an equal volume of the W2 solution to form a coarse secondary emulsion. A peristaltic pump was used to pump the coarse emulsion through a porous sintered metal filter element with 7 μm nominal pore size. The emulsion was recirculated through the element for approximately 10 minutes until the average droplet size was less than 10 microns. Examination of the secondary emulsion under a microscope showed discrete organic droplets containing much smaller blue droplets within.

The emulsion was diluted into an aqueous bath containing 0.25% ethyl dimethylamino-propyl carbodiimide at 25° C. that was osmotically adjusted with dextrose to 300 mOs/kg. After 5 minutes, poloxamer 188 surfactant was dissolved into the aqueous bath at a concentration of 0.25% to inhibit aggregation of the emulsion droplets. A 50 ml sample of the bath was centrifuged at 500 rpm for 10 minutes. The emulsion microdroplets were retrieved by centrifugation and rinsed with a 0.5% solution of poloxamer 188 and osmotically adjusted to 300 mOs/kg using dextrose. The organic-polymer droplets were retrieved, formulated in an osmotically balanced solution containing cryoprotectants and bulking agents, and lyophilized. When the lyophilization cycle was complete, the lyophilization chamber was filled with nitrogen gas to a pressure of slightly less than atmospheric and the vials were then stoppered.

Examination of the reconstituted microparticles under the microscope revealed discrete gas filled microparticles.

### EXAMPLE 4

### Destruction of Microparticles Using Ultrasound

An experimental apparatus was assembled and operates as described herein. A reservoir containing 50 ml of deionized water is continuously stirred with a magnetic stir bar. A peristaltic pump draws water from the bottom of the reservoir and pumps it through a ⅛ inch diameter tube. The tube is fitted with a Y connector that shunts a small volume of the flow through a 200 μm diameter cellulose tube. The tube is suspended in a glass bottom tank filled with water and sized to fit on a microscope stage. The microscope is equipped with long working distance objectives and condenser. The cellulose tube is positioned so that a portion of the tube passes through the optical focus of the microscope in a level plane. An HP S4 ultrasound probe is mounted in the side wall of the tank and connected to an HP 5500 ultrasound scanner.

A single air bubble was attached to the cellulose tube at the optical focus of the microscope. The position was verified by looking through the microscope and viewing the air bubble at low magnification. Using micro-positioners, the acoustic focus of the ultrasound probe was positioned to focus on the cellulose tube at the optical focus of the microscope. The location of the acoustic focus was set by adjusting the ultrasound transducer in the X and Y axes until the maximum ultrasound signal was returned. Signal intensity was determined using the 256 gray scale image intensity on the HP 5500 video monitor.

Microparticles manufactured in a manner described in Example 1 were tested in accordance with the following procedure. A single vial of microparticles was reconstituted with 3 ml of deionized water and agitated to dissolve the lyophilized cake. A 1 ml aliquot was diluted into the 50 ml reservoir of deionized water. The peristaltic pump was started and the flow was adjusted until the microparticles were seen flowing through the cellulose tube under the microscope. The HP 5500 ultrasound scanner was turned on and set to emit a series of 5 pulses at 1.8 Mhz and a mechanical index of 0.8. Closing a valve stopped the flow of microparticles inside the cellulose tube. The ultrasound machine was triggered and the microparticles were observed to rapidly flood. Some were seen to fragment before flooding. Flooding is evidenced by a change in appearance from an opaque easily viewed microparticle to one which is nearly transparent. The microparticles were verified as flooded based on their buoyancy. Gas filled microparticles floated to the top of the tube while flooded microparticles sank to the bottom.

### EXAMPLE 5

### Release of Bromophenol Blue Dye from Microparticles Upon Insonation

Two vials of lyophilized microparticles encapsulating bromophenol blue dye and prepared in a manner similar to the procedure described in Example 3 were reconstituted with 5 mL DI water. The contents of the two vials were combined and the suspension was allowed to stand for approximately 1 hour. Using a needle and syringe, approximately 9 ml of the subnatant was removed and discarded. The microparticles which had floated to the top were resuspended in 10 ml of DI water. The suspension was divided into two samples and each was allowed to again stand for 1 hour. From the first sample 1.5 ml supernatant was carefully withdrawn using a syringe and filtered through a 0.45 micron syringe filter. The second sample was resuspended with gentle mixing and placed in a 300 ml water bath. The bath was insonated using a Virtis VirSonic Homogenizer at a setting of 8 for 1 minute. The microparticles are known to flood at this setting and duration. After insonation the suspension was filtered through a 0.45 micron syringe filter. Using a Beckman DU 640 spectrophotometer, both filtered solutions were scanned from 450 nm to 700 nm wavelength.

A comparison of the absorbance measurement vs. wavelength shows an increase in the concentration of bromophenol blue in the insonated solution. Results shown in FIG. 2 demonstrate a release of dye from the loaded microparticles resulting from exposure to ultrasound.

### EXAMPLE 6

### Encapsulation of Adenosine Triphosphate in a Polymer-Matrix Microparticle

A solution of 0.5% PVA and 10 mmol imidazole was prepared. Adenosine triphosphate (ATP) was added to the solution at a 2.5% concentration. The pH of the ATP solution was adjusted to 6.8 using 1N NaOH. The osmolality of the ATP solution was measured with a calibrated osmometer and recorded at 180 mmol/kg. This solution will be referred to as the W1 solution. A 5% (wt/wt) solution of poly(DL-lactide) was made by dissolving the polymer in a 50:50 blend of p-xylene and ethyl acetate. A water-in-oil emulsion was created using a Virtis Virsonic 20 kHz ultrasound probe. One part ATP solution was pipetted slowly into 4 parts polymer solution while sonicating at power level 5. The ultrasound probe power was increased to level 9 after all of the ATP solution was added. Microscopic examination of this primary emulsion revealed sub-micron size water droplets that were well dispersed throughout the emulsion.

An outer water phase, W2, consisting of a 1% PVA solution at pH 6.8 was prepared. The osmolality of this W2 solution was adjusted using glycine to match the osmolality of the inner W1 phase of 180 mmol/kg. The W2 solution was placed in a 250 ml jacketed tempering beaker maintained at 20° C. and agitated slowly with a stir bar. The primary water-in-oil emulsion was slowly added to an equal volume of the W2 solution to form a coarse double emulsion. A peristaltic pump was used to pump the coarse emulsion through a porous sintered metal filter element having 7 micron nominal pore size. The double emulsion was recirculated through the filter element for approximately 10 minutes. Examination of the secondary emulsion under a microscope showed discrete organic drops containing microdroplets within.

The emulsion was added to a water rinse bath at a 1:10 ratio w/w. The pH 6.8 rinse bath was adjusted with glycine to an osmolality of 180 mmol/kg and held at a constant temperature of 30° C. in a 600 ml jacketed tempering beaker. The ethyl acetate was allowed to evaporate from the rinse bath for 1 hour. A 40 ml sample of the bath was removed and centrifuged at 2000 rpm for 10 minutes. The concentrated microdroplets were separated from the underlying liquid and then lyophilized in 10 ml vials in an osmotically balanced aqueous medium. When the lyophilization cycle was complete, the lyophilization chamber was filled with nitrogen gas to a pressure of slightly less than atmospheric and the vials were then stoppered.

Observation of the reconstituted microparticles under a microscope showed the particles were gas-filled and roughly spherical. The majority of the particles was estimated to be less than 10 microns diameter. The bubbles were observed to readily float confirming that they were gas-filled. The internal regions of the particles were not clearly visible. The particles were observed to remain air filled 72 hours following reconstitution.

### EXAMPLE 7

### Release of Adenosine Triphosphate from Microparticles Following Insonation

Adenosine triphosphate release was evaluated using microparticles manufactured as described in Example 6. Two vials were each reconstituted with 10 ml of deionized water and then transferred to a 50 ml centrifuge tube. The centrifuge tube was swirled to achieve a uniform suspension of microparticles. The suspension was divided equally into two labeled 15 ml centrifuge tubes. Both tubes were allowed to stand undisturbed for 1 hour. One tube was selected and placed in a water bath. The water bath was insonated at 20 kHz for 30 seconds with a Virtis ultrasonic homogenizer for 30 seconds at power level 9.

A small sample of the insonated microparticles was inspected under a microscope and observed to be semi-transparent indicating that they had become flooded. The insonated tube was allowed to stand for 30 minutes to allow the flooded particles to settle. A 5 ml sample of supernatant was withdrawn from the center of both the insonated and non-insonated control vials using a needle and syringe. The samples were filtered through a 0.45 micron syringe filter into separate labeled centrifuge tubes.

A 100 fold dilution of each filtered sample was prepared for analysis by UV-Vis spectrophotometry. The absorbance of each sample was determined at 260 nm in a quartz cuvette. The absorbance of the non-insonated preparation was 0.0736 while the absorbance of insonated sample was 0.1577. These values represent a 53.3% increase in free ATP in the insonated sample compared to the non-insonated sample. From this data, it was determined that 1.5 mg of encapsulated adenosine triphosphate is contained in each vial.

### EXAMPLE 8

### Encapsulation of Adenosine Triphosphate in a Dual Polymer Matrix Microparticle

A solution of 1.0% w/w ATP, 6% human serum albumin, and 5 mM Tris (pH 7) was prepared (W1). Separately, a 6% solution of a 6:4 mixture of poly DL-lactide-coglycolide and poly DL-lactide dissolved in a 4:6 mixture of dioxane and p-xylene was prepared. A 4 gm portion of W1 was added to 20 gm polymer solution while ultrasonically agitating the mixture using a Virtis Versonic ultrasonic homogenizer at a power level of 7 for 20 seconds. Microscopic inspection of the primary emulsion revealed sub-micron sized water droplets that were well dispersed throughout the emulsion.

A 25 gm solution of 1% polyvinyl alcohol and 2.8% mannitol was prepared (W2) and kept at a constant 15° C. To this was added the previously prepared primary emulsion and the mixture was circulated through a sintered metal filter element having an average pore size of 7 microns. After approximately 3 minutes an additional 100 gm of a 2.8% solution of mannitol was added. Circulation through the filter element continued for another minute. Microscopic examination of the resulting secondary emulsion revealed discrete droplets containing much smaller droplets therein.

A portion of the prepared emulsion droplets were washed by centrifugation, resuspended in a 2.8% solution of mannitol, dispensed in 10 ml vials and lyophilized. The lyophilized product was reconstituted with DI water. Microscopic examination of the microparticles revealed that they were gas filled.

### EXAMPLE 9

### Release of Adenosine Triphosphate from Dual Polymer Microparticles Following Insonation

ATP release was evaluated using microparticles manufactured as described in Example 8. Two vials were reconstituted with 10 ml of deionized water and then transferred to a 15 ml centrifuge tube. One tube was placed in a water bath and the bath insonated for 30 seconds with a Virtis ultrasonic homogenizer at a power setting of 8. The second tube was retained as a control.

A 3 ml aliquot was withdrawn from both tubes and then filtered through a 0.2 micron syringe filter. The samples were analyzed by UV-Vis spectrophotometry to establish relative amounts of ATP in solution. The absorbance of the non-insonated preparation was 0.573 while the absorbance of the insonated sample was 0.849 indicating that while there was an initial release of ATP upon reconstitution, additional ATP was delivered into solution following insonation of the microparticles.

What is claimed is:

1. A microparticle composition for delivery of a pharmaceutical agent by ultrasound triggering comprising microparticles having a porous, gas-containing interior polymer matrix, and a plurality of cavities dispersed within said matrix, wherein said cavities contain a gas and said pharmaceutical agent.

2. A microparticle composition according to claim 1 further comprising an outer shell wherein said shell comprises a different polymer from the polymer comprising said polymer matrix.

3. A microparticle composition according to either claim 1 or 2 further comprising an outer layer of a biologically compatible amphiphilic material.

4. A microparticle composition according to claim 1 wherein said polymer matrix comprises a polymer selected from the group consisting of polymers or copolymers of two or more of polylactide, polyglycolide, polycaprolactone, polyhydroxybutyrate, polyhydroxyvalerate, polyalkylcyanoacrylates, polyamides, polydioxanones, poly-beta-aminoketones, polyanhydrides, poly(ortho)esters, polyamine acids and copolymers of lactides and lactones.

5. A microparticle composition according to claim 4 wherein said polymer matrix comprises polylactide.

6. A microparticle composition according to claim 2 wherein said outer shell comprises a polymer selected from the group consisting of polymers or copolymers of polylactide, polyglycolide, polycaprolactone, polyhydroxybutyrate, polyhydroxyvalerate, polyalkylcyanoacrylates, polyamides, polydioxanones, poly-beta-aminoketones, polyanhydrides, poly(ortho)esters, polyamine acids and copolymers of lactides and lactones.

7. A microparticle composition according to claim 6 wherein said outer shell comprises polylactide-co-glycolide, a copolymer of polylactide and polyglycolide.

8. A microparticle composition according to claim 3 wherein said biologically compatible amphiphilic material is selected from the group consisting of gelatin, albumin, globulins, casein, and collagen.

9. A microparticle composition according to claim 8 wherein said outer layer comprises albumin.

10. A microparticle composition according to claim 1 wherein said gas is selected from the group consisting of air, nitrogen, oxygen, argon, helium, carbon dioxide, xenon, a sulfur halide, a halogenated hydrocarbon, and combinations thereof.

11. A microparticle composition according to claim 10 wherein said gas comprises nitrogen.

12. A microparticle composition according to claim 1 wherein said microparticles have diameters within the range of 1 to 1000 microns.

13. A microparticle composition according to claim 12 wherein said microparticles have diameters within the range of 1 to 10 microns.

14. A microparticle composition according to claim 3 wherein said microparticles are of a size capable of passing through the capillary circulation and comprise surface targeting moieties for binding to selected tissues.

15. A method of forming a microparticle composition suitable for delivering a pharmaceutically active agent by ultrasonic triggering comprising the steps of:
a. forming a first emulsion from a first aqueous phase comprising said pharmaceutically active agent and an organic solvent phase substantially immiscible with said aqueous phase comprising a first solvent and a polymer;
b. forming a second emulsion from said first emulsion and a second aqueous phase, said second emulsion comprising droplets containing said organic solvent phase and further containing a plurality of microdroplets of said first aqueous phase,
c. removing said first solvent from said organic solvent phase and water from said first aqueous phase to form microparticles having a porous gas-containing interior polymer matrix and a plurality of cavities dispersed within said matrix.

16. A method according to claim 15 wherein said organic solvent phase further comprises a second solvent and a second polymer soluble in the mixture of said first solvent and said second solvent and insoluble in said first solvent, further comprising the step after step b) of removing said second solvent to form an outer shell comprising said second polymer on said microparticles.

17. A method according to claim 15 wherein said second aqueous phase comprises a biologically compatible amphiphilic material, further comprising the step after step b) of diluting said second emulsion with an aqueous bath containing a chemical cross-linking agent to form an outer layer on said microparticles.

18. A method according to claims 15 or 17 wherein said first solvent and said water are removed by lyophilization.

19. A method according to claim 16 wherein said first solvent and said water are removed by lyophilization.

20. A method for delivery of a pharmaceutical agent to a region of interest within a fluid filled cavity, vessel, or fluid perfused tissue by ultrasound triggering comprising the steps of:
a. introducing a microparticle composition according to claims 1 or 2 into said region of interest,
b. applying an ultrasound signal to said region of interest at a power intensity sufficient to induce rupture of said microparticles,
c. maintaining said power intensity until at least a substantial number of the microparticles are ruptured.

21. A method according to claim 20 comprising, after step a) the step of monitoring the location of said microparticles within said cavity, vessel, or fluid perfused tissue by applying an ultrasound signal to said region of interest at a power intensity below that which is sufficient to rupture said microparticles.

22. A method according to claim 20 wherein said ultrasound power intensity sufficient to induce rupture of said microparticles is at a mechanical index between about 0.1 and about 1.9.
